(19) United States
(12) Patent Application Publication
(10) Pub. No.: US 2011/0054427 A1
(43) Pub. Date: Mar. 3, 2011

(54) MALE URINARY INCONTINENCE DEVICE AND METHOD

(76) Inventors: Myles Kobren, Hicksville, NY (US); Richard Malerba, Coram, NY (US); Ted Felderman, Syosset, NY (US)

(21) Appl. No.: 12/549,133

(22) Filed: Aug. 27, 2009

(51) Int. Cl. A61F 5/44; A61F 6/04

(52) U.S. Cl. 604/349; 128/844

(57) ABSTRACT

A male urinary incontinence system includes a first portion that fit over a penis and has a distal end with a fluid opening. A urine collection vessel having a proximal end removably attaches to the distal of the first portion, thereby allowing a user to urinate through the fluid opening when the collection vessel is not attached. The first portion may a flexible, fluid-impermeable sheath such as a latex condom. The collection vessel may attach to the first portion through a threaded connection. The collection vessel preferably contains an absorbent material, more preferably a superabsorbent material such as a polyacrylate. The collection vessel may also assume the form of a flexible, fluid-impermeable sheath such as latex condom, enabling the absorbent or superabsorbent material to swell during the urine collection process.

### FIELD OF THE INVENTION

This invention related generally to incontinence collection devices and, in particular, to a two-part male urinary incontinence device.

### BACKGROUND OF THE INVENTION

Urinary incontinence is the accidental release of urine. Incontinence can occur for a variety of reasons. If the bladder contracts at the wrong time, or if it squeezes too hard, urine may leak out. If the muscles around the urethra are damaged or weak, urine can leak out even in the absence of a bladder problem. If the bladder gets too full, or if something is blocking the urethra, urine can build up in the bladder and cause leaking. Urinary incontinence happens more often in older men than in younger men, but it is not just a normal part of aging.

Urinary incontinence can be short-term or long-lasting (chronic). Short-term incontinence is often caused by other health problems or treatments. Chronic urinary incontinence can also be due to a number of factors, including stress, muscle weakness, or blockages related to an enlarged prostate or a narrow urethra.

Treatments are different for each person. They depend on the type of incontinence and how much it affects the individual's life. Treatment may include medicines, simple exercises, or both. A few men need surgery, but most do not. Various diaper-type products exist such as guards and pads, but they can leak, overflow and cause odor.

### SUMMARY OF THE INVENTION

This invention addresses male urinary incontinence problems with a two-part system that is discrete and easy to use. A first portion, that fit over a penis, has a distal end with a fluid opening. A urine collection vessel having a proximal end removably attaches to the distal of the first portion, thereby allowing a user to urinate through the fluid opening when the collection vessel is not attached.

In the preferred embodiment, the first portion is a flexible, fluid-impermeable sheath such as a latex condom. The collection vessel may attach to the first portion through a threaded connection. The collection vessel preferably contains an absorbent material, more preferably a superabsorbent material such as a polyacrylate. The collection vessel may also assume the form of a flexible, fluid-impermeable sheath such as latex condom, enabling the absorbent or superabsorbent material to swell during the urine collection process.

### BRIEF DESCRIPTION OF THE DRAWING

FIG. 1 is an exploded view of the preferred embodiment of the invention.

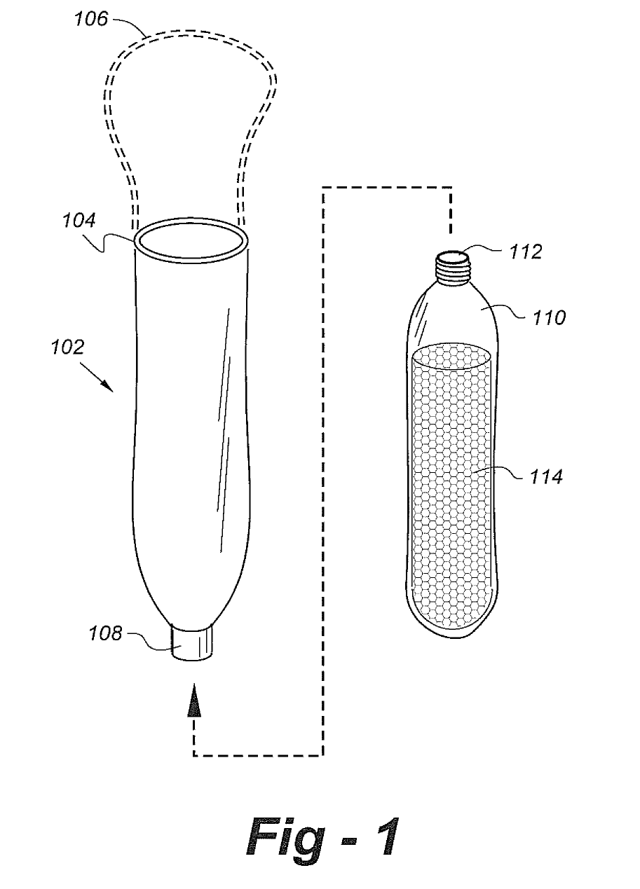

### DETAILED DESCRIPTION OF THE INVENTION

This invention provides a solution to male urinary incontinence which is discrete and easy to use. Referring to the FIGURE, the invention uses a two-part system including a first portion 102 that fits over the penis and a collection vessel 110 that collects expelled urine. In the preferred embodiment, the collection vessel has a threaded end 112 that screws into the distal end 108 of the first portion 102. Other attachment mechanisms may alternatively be used, including bayonet and quick connect/disconnect connections.

Also in the preferred embodiment, the first portion 102 is flexible, fluid-impermeable sheath such as a latex condom which is held in position through a frictional fit and/or the use of a band 104 or optional strap(s) 106. The end 108 is a plastic piece that is bonded and sealed to the flexible sheath.

The collection vessel 102 may simply be empty if the incontinence problem is not that pronounced, or it may contain an absorbent material such tissue paper, cotton, sponge, or fluff pulp. In the preferred embodiment, however, the vial 102 contains a superabsorbent material such as polyacrylate or other superabsorbent polymer. Such polymers are made from the polymerization of acrylic acid blended with sodium hydroxide in the presence of an initiator to form a polyacrylic acid sodium salt (sometimes referred to as sodium polyacrylate). Other materials are also used to make a superabsorbent polymer, such as polyacrylamide copolymer, ethylene maleic anhydride copolymer, cross-linked carboxy-methyl-cellulose, polyvinyl alcohol copolymers, cross-linked polyethylene oxide, and starch grafted copolymer of polyacrylonitrile.

The polyacrylate 114 or other material absorbs urine, forming a flexible spongy mass encased in the collection vessel 110. Thus, while the collection vessel 110 may be made of a rigid or semi-rigid plastic material, in the preferred embodiment it is also flexible and condom-like, enabling absorbent material—particularly superabsorbent material—to swell during the collection process. Like the first portion 102, the collection vessel may be a latex sheath or other flexible, fluid impermeable material bonded to a harder plastic end 112 to provide threads or other connection mechanism.

A distinct advantage of the invention is that the collection device 110 may be easily removed after use as determined by the user and placed in a waste receptacle with the first portion 102 remaining in position. This allows the user to detach the vial 110 and urinate if needed, after which the same or a different vial may be attached.

We claim:

1. A male urinary incontinence device, comprising:
a first portion that fit over a penis, the first portion having a distal end with a fluid opening; and
a urine collection vessel having a proximal end that removably attaches to the distal of the first portion, thereby allowing a user to urinate through the fluid opening when the collection vessel is not attached.

2. The male urinary incontinence device of claim 1, wherein the first portion is a flexible, fluid-impermeable sheath.

3. The male urinary incontinence device of claim 1, wherein the first portion is a latex condom.

4. The male urinary incontinence device of claim 1, wherein the collection vessel is attached to the first portion through a threaded connection.

5. The male urinary incontinence device of claim 1, wherein the collection vessel contains an absorbent material.

6. The male urinary incontinence device of claim 1, wherein the collection vessel contains a superabsorbent material.

7. The male urinary incontinence device of claim 1, wherein the collection vessel contains a polyacrylate.

8. The male urinary incontinence device of claim 1, wherein the collection vessel is a flexible, fluid-impermeable sheath.

9. The male urinary incontinence device of claim 1, wherein the collection vessel is a latex condom.

10. A male urinary incontinence device, comprising:
a flexible, fluid-impermeable sheath that fit over a penis, the sheath having a distal end with a fluid opening;
a flexible, fluid-impermeable urine collection vessel having a proximal end that removably attaches to the distal of the first portion; and
an absorbent or superabsorbent material disposed with the collection vessel.

12. The male urinary incontinence device of claim 10, wherein the flexible, fluid-impermeable sheath is a latex condom.

13. The male urinary incontinence device of claim 10, wherein the collection vessel is attached to the flexible, fluid-impermeable sheath through a threaded connection.

14. The male urinary incontinence device of claim 10, wherein the collection vessel contains a polyacrylate.

15. The male urinary incontinence device of claim 10, wherein the collection vessel is a latex condom.
